(19) United States
(12) Patent Application Publication
(10) Pub. No.: US 2011/0160511 A1
(43) Pub. Date: Jun. 30, 2011

(54) Gas adsorption and gas mixture separations using porous organic polymer

(75) Inventors: Joseph T. Hupp, Northfield, IL (US); Chad A. Mirkin, Wilmette, IL (US); Omar K. Farha, Morton Grove, IL (US); Alexander M. Spokoyny, Chicago, IL (US)

(73) Assignee: Northwestern University

(21) Appl. No.: 12/927,689

(22) Filed: Nov. 19, 2010

Related U.S. Application Data
(60) Provisional application No. 61/283,034, filed on Nov. 25, 2009.

(51) Int. Cl. C07C 7/12; C08G 73/10

(52) U.S. Cl. 585/823; 521/189

(57) ABSTRACT

A method of separating a mixture of carbon dioxide and methane using a porous organic polymer material which includes non-planar monomeric building blocks linked by imide linkers wherein the polymer material selectively absorbs CO₂. The polymer material can be chemically reduced to increase its selectivity toward CO₂.

### RELATED APPLICATION

This application claims benefits and priority of provisional application Ser. No. 61/283,034 filed Nov. 25, 2009, the disclosure of which is incorporated herein by reference.

### CONTRACTUAL ORIGIN OF THE INVENTION

This invention was made with government support under Grant No. EEC-0647560 awarded by the National Science Foundation-NSEC. The government has certain rights in the invention.

### FIELD OF THE INVENTION

The present invention relates to methods and materials for adsorption of gases such as carbon dioxide in the separation of carbon dioxide and methane.

### BACKGROUND OF THE INVENTION

Carbon dioxide is often found as an impurity in natural gas and landfill gas, where methane is the major component. The presence of CO₂ reduces the energy content of natural gas and can lead to pipeline corrosion. If natural gas meets established purity specifications, it is designated “pipeline quality methane,” which increases its commercial value. To meet pipeline requirements, natural gas must comply with strict CO₂ concentration limits, as low as 2%.

Elimination of contaminant carbon dioxide from natural gas and landfill gas streams, composed mostly of methane, thus is an important problem. The presence of CO₂ in natural gas significantly lowers the energy density of the gas stream and can lead to pipeline corrosion over time. Current technologies for separation of CO₂ from CH₄ include cryogenic distillation, membrane separation, chemical absorption, and physical adsorption. The pressure swing adsorption (PSA) method is of particular industrial interest for its outstanding energy efficiency and low operating costs.

The fundamental component of any PSA system is a highly selective CO₂ adsorbent that can accommodate large quantities of the gas and is easily regenerated. Separations with porous materials such as zeolites and activated carbons have been widely explored. More recently, new classes of materials such as metal-organic frameworks (MOFs), covalent organic frameworks (COFs), and porous polymers have shown a propensity for selective gas adsorption. These microporous solid materials have also shown promise in gas storage and catalytic applications” in addition to their gas separation capabilities.

Low-density microporous solids have garnered considerable recent attention. For example, Yaghi and co-workers in Science, 2002, 295, 469-472, in particular, have made pioneering contributions to the development of these materials with their work on metal-organic frameworks (MOFs) and, more recently, two- or three-dimensional covalent organic frameworks (COFs) (Science, 2005, 310, 1166-1170).

Both classes of materials are crystalline polymers and both are permanently microporous. In a recent work. Mirkin et al. in Nature, 2005, 438, 651-654, have shown that by arresting the growth of a coordination polymer at early stages, one can create nano- or microparticles. These particles typically lack crystallinity, but nevertheless retain good permeability and porosity with respect to both ions and gases. From these studies, one can conclude that apparent crystallinity is not a requirement for permanent microporosity in coordination polymers. Indeed, several examples of noncrystalline “polymers of intrinsic microporosity” have already been reported, most notably by McKeown and co-workers (Chem. Soc. Rev., 2009, 35, 675-683). Although the majority are one-dimensional, some are network polymers. Microporosity is achieved mainly by utilizing twisted (spiro type) monomers. Thomas and coworkers (Macromolecules, 2008, 41, 2880-2885), for example, recently utilized spirobifluorene to produce porous polyimide and polyamide materials, suitable for hydrogen storage)

### SUMMARY OF THE INVENTION

The present invention provides in one embodiment a method for selectively adsorbing carbon dioxide in the separation of carbon dioxide and methane using a porous organic polymer material having three dimensional (non-planar) building blocks linked by imide linkers. The method is useful to separate carbon dioxide from a mixture of carbon dioxide and methane by contacting the gas mixture and the porous polymer material that selectively adsorbs carbon dioxide from the mixture. The invention is advantageous for the selective removal of carbon dioxide from natural gas, landfill gas, and other gas mixtures of CO₂ and CH₄.

In an illustrative embodiment of the invention, the polymer material useful in practice of the method includes tetrahedral tetra-amino building blocks linked by napthalene dianhydride (diimide) linkages. This material selectively adsorbs carbon dioxide from a room temperature mixture of carbon dioxide and methane and is especially effective to this end at relatively low bulk gas pressures and high mole fractions of methane in the mixture.

Another embodiment of the invention envisions chemically reducing the polymer material to increase its selectivity to carbon dioxide. For example, the porous polymer described above can be reduced with alkali metal to this end.

The invention also envisions a method of making the porous organic polymer wherein the polymer is made by condensation of a non-planar monomer (building blocks) with imide monomer (linkers). The non-planar monomer can be tetra-amine. The imide monomer can be napthalene dianhydride.

Other advantages and features of the present invention will become apparent from the following detailed description taken with the following drawings.

### BRIEF DESCRIPTION OF THE DRAWINGS

FIG. 1 is a diagram for synthesizing a porous polyimide-based polymer material pursuant to an embodiment of the invention made by Scheme 1 where (i) involves HNO₃ (fuming), Ac20/AcOH, rt. 50% and (ii) involves Raney Ni/ThIF, reflux, 72%, and (iii) involves DMF/propionic acid.

FIG. 2a is a TGA trace of compound 5 (bottom trace), 6 (top trace), and resolvated 6 (middle trace). FIG. 2b is a nitrogen isotherm at 77K.

FIG. 3a illustrates CO₂ and CH₄ isotherms of polymer 6 at 298 K. FIG. 3b illustrates selectivity of CO₂ and CH₄ at different pressures and mole fractions.

FIG. 4 shows pore size distribution of polymer 6 obtained by Horvath-Kawazoe (HK) method from the nitrogen isotherm.

FIG. 5 shows accumulative pore volume of 6 using the HK method from nitrogen isotherm.

FIG. 6 shows pore size distribution of 6 obtained from CO₂ isotherm.

FIGS. 7a, 7b, and 7c show surface area determination data for polymer 6 in the various conditions indicated.

FIGS. 8a and 8b are SEM's of polymer 5 and 6, respectively.

FIG. 9 are ¹³C CP-MAS spectrums of polymer 5 (bottom) and polymer 6 (top).

FIG. 10 is a diagram for chemical reduction of the porous polyimide-based polymer material pursuant to another embodiment of the invention using Scheme 1′.

FIGS. 11A1, 11A2, 11A3 are measured CO₂ and CH₄ isotherms at 298 K along with the dual-site LangmuirFreundlich fits for as-synthesized 4 (FIG. 11A1), Li_{0.35} reduced 5 (FIG. 11A2), and Li_{0.55} reduced 6 (FIG. 11A3).

FIGS. 11B1, 11B2, 11B3 are IAST selectivity of CO₂ versus CH₄ at various pressures and mole fractions of CH4 (y_{CH4}) for as-synthesized 4 (FIG. 11B1), Li_{0.35} reduced 5 (FIG. 11B2), and Li_{0.55} reduced 6 (FIG. 11B3).

FIG. 12 shows normalized isotherm data for CO₂ (closed symbols) and CH₄ (open symbols).

### DETAILED DESCRIPTION OF THE INVENTION

An embodiment of the present invention provides a method for selectively adsorbing carbon dioxide in the separation of carbon dioxide and methane using a porous organic polymer material. In an illustrative embodiment of the invention, the porous polymer material comprises a three dimensional structure made up of three dimensional (non-planar) monomeric building blocks linked by imide linkages.

The method is useful to separate carbon dioxide from a mixture of carbon dioxide and methane by contacting the gas mixture and the porous polymer material that selectively adsorbs carbon dioxide from the mixture. The polymer material can selectively adsorb carbon dioxide from a room temperature mixture of carbon dioxide and methane and is especially effective to this end at relatively low bulk gas pressures and high mole fractions of methane in the mixture. The invention is advantageous for the selective removal of carbon dioxide from natural gas, landfill gas, and other gas mixtures of CO₂ and CH₄.

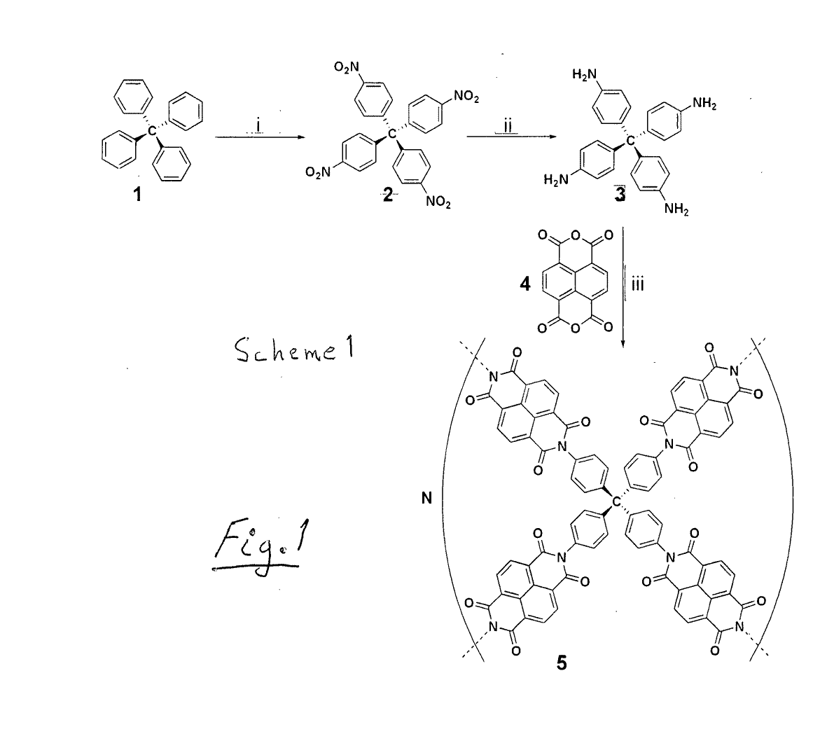

The method can be practiced pursuant to an illustrative embodiment of the invention using a porous organic polymer material that comprises a three dimensional structure made up of three dimensional tetrahedral tetra-amino building blocks (polyhedral building blocks) linked by napthalene dianhydride (diimide) linkages. The polymer material can be made by condensation of cheap and abundant amine-bearing monomers and anhydride-bearing monomers. In particular, the polymer material (5) can be made by from the condensation of tetrahedral tetra-amino building blocks (3) with napthalene dianhydride (4) in dimethylformamide (DMF) as illustrated in FIG. 1 for synthesis Scheme 1 and as described below. The desired micro- and ultramicro-porosity is engendered by using the tetrahedral building block that is effective to produce a three-dimensional (locally diamondlike) network. Additionally facilitating porosity, by inhibiting efficient packing of any catenated regions, should be the large dihedral angle (nominally 90°) between the phenyl and diimide subunits of (polymer 5).

### Example 1

For purposes of illustration and not limitation, practice of the invention will be illustrated using polymer (5) illustrated in FIG. 1.

### Synthesis and Testing of Microporous Organic Polymer (5):

Starting materials were purchased from Sigma-Aldrich (ACS grade) and used without further purification unless otherwise noted. 1 was purchased from Alfa Aesar and used as received. Deuterated d⁶-DMSO was acquired from Cambridge Isotopes Inc. and used as received. ¹H and ¹³C NMR spectra were collected on a Varian Mercury 300 and referenced to a residual solvent peak. Elemental analyses (C, H and N) were performed by Quantitative Technologies (Intertek), Whitehouse, N.J. Thermogravimetric analyses (TGA) were performed on a Mettler-Toledo TGA/SDTA851e. ¹³C CPMAS NMR was performed on a Varian Inova 400 Widebore instrument. FT-IR measurements were done on a Perkin-Elmer 100 spectrometer equipped with a diamond ATR unit.

Low-pressure hydrogen and nitrogen adsorption measurements were performed using an Autosorb 1-MP from Quantachrome Instruments as described in Farha et al. U.S. Pat. No. 7,744,842. Ultra-high purity grade H₂ and N₂ were used for all adsorption measurements. Samples of 4 were the loaded into a sample tube of known weight and activated at room temperature and dynamic vacuum for about 24 hours to completely remove guest solvents. After activation, the sample and tube were re-weighed to obtain the precise mass of the evacuated sample. N₂ adsorption isotherms were measured at 77K (liquid N₂ bath) and H₂ adsorption isotherms were measured at 77 and 87K (liquid N₂ and Ar bath respectively).

The adsorption isotherms of CO₂ and CH₄ on the sample were measured volumetrically at 298 K up to 18 atm. The void volume of the system was determined by using He gas. CO₂ (99.9%) and CH₄ (99%) were obtained from Airgas Inc. (Radnor, Pa.). Prior to analysis, gases were passed through molecular sieves to remove residual moisture. Equilibrium pressures were measured with an MKS Baratron transducer 627B (accuracy±0.12%). Adsorbate was dosed into the system incrementally, and equilibrium was assumed when no further change in pressure was observed (within 0.01 kPa).

Compound 2 of FIG. 1 was synthesized in a manner adopted from: Ganesan, P.; Yang, X.; Loos, J.; Savenije, T. J.; Abellon, R. D.; Zuilhof, H.; Sudholter, E. J. R. J. Am. Chem. Soc. 2005, 127, 14530-14531, the teachings of which are incorporated herein by reference. In particular, five (5) grams (15.6 mmoles) of compound 1 was slowly added to 25 ml of fuming nitric acid, while being vigorously stirred on ice/salt water bath (about −5° C.). To the formed suspension, approximately 25 mL of 1:2 mixture of acetic anhydride (Ac₂O) and glacial acetic acid (AcOH) was slowly added, and stirred for 15 minutes at −5° C. Additional 80 mL of AcOH was then added and the suspension was stirred for 5 minutes. The precipitate was then filtered on a glass frit, washed with AcOH (2×, 100 mL), followed by methanol (2×, 100 mL) and chilled tetrahydrofuran (2×, 50 mL) and subsequently dried in vacuo, to afford a yellowish solid.* (3.9 grams, 50%) ¹H NMR (300 MHz, d⁶-DMSO, 25° C.): δ 8.2 (d, 8H), δ 7.6 (d, 8H); ¹³C {¹H} NMR (75.5 MHz, d⁶-DMSO, 25° C.): δ 151.7 (s), 146.8 (s), 132.2 (s), 124.5 (s), 67.7 (s).

THF forms an inclusion compound with compound 2, which is observed via NMR. (See: Thaimattam, R.; Xue, F.; Sarma, J. A. R. P.; Mak, T. C. W.; Desiraju, G. R. J. Am. Chem. Soc. 2001, 123, 4432-4445.)

Compound 3 was synthesized in a manner adopted from: Yang, X.; Loos, J.; Savenije, T. J.; Abellon, R. D.; Zuilhof, H.; Sudholter, E. J. R. J. Am. Chem. Soc. 2005, 127, 14530-14531. In particular, twenty (20) grams of Raney Ni were added to 3 grams of compound 2 (6 mmoles) dissolved in 200 mL of tetrahydrofuran (THF), while being stirred under nitrogen. To the reaction slurry, 4 grams of hydrazine hydrate (N₂H₄×H₂O) was slowly added via syringe. The reaction was refluxed for 4 hours, and then filtered while hot. The solid residue was washed with ethanol, and all filtrate fractions were combined and dried in vacuo. The crude product was washed with ethanol (100 mL) to afford analytically pure 3 as white solid (1.65 grams, 72%). ¹H NMR (300 MHz, d⁶-DMSO, 25° C.): δ 6.63 (d, 8H), δ 6.34 (d, 8H) δ 4.81 (bs, 8H); ¹³C {¹H} NMR (75.5 MHz, d⁶-DMSO, 25° C.): δ 146.3 (s), 136.5 (s), 131.7 (s), 113.1 (s), δ 61.8 (s).

Compound (polymer) 5 was synthesized by dissolving 70 mg (26 mmol) 1,4,5,8-napthalene-tetracarboxylic dianhydride in 10 mL DMF and heating with stirring in a 170° C. oil bath. Once refluxing, a solution of 50 mg (13 mmol) of 3 in 5 mL propionic acid was added dropwise. When the light brown mixture became cloudy, 5 mL DMF were added and a tan precipitate soon formed. The solution was stirred at 170° C. for 20 hours, then filtered and washed with DMF to yield a tan, fluffy powder (135 mg).

Polymer 6 involved evacuating polymer 5 while heating at 160° C. for 24 hours. Elemental analysis: calculated; C, (75.35); H, (2.86); N, (6.63); and found; C, (71.55); H, (3.01); N, (7.30).

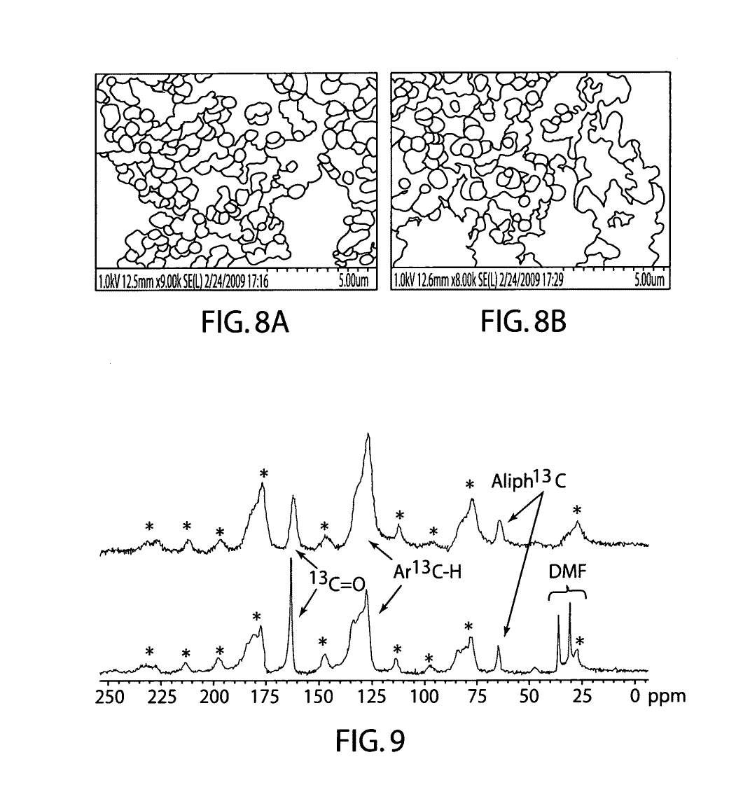

FIGS. 8a and 8b are SEM's of polymer 5 and 6, respectively. FIG. 9 are ¹³C CP-MAS spectrums of polymer 5 (bottom) and polymer 6 (top).

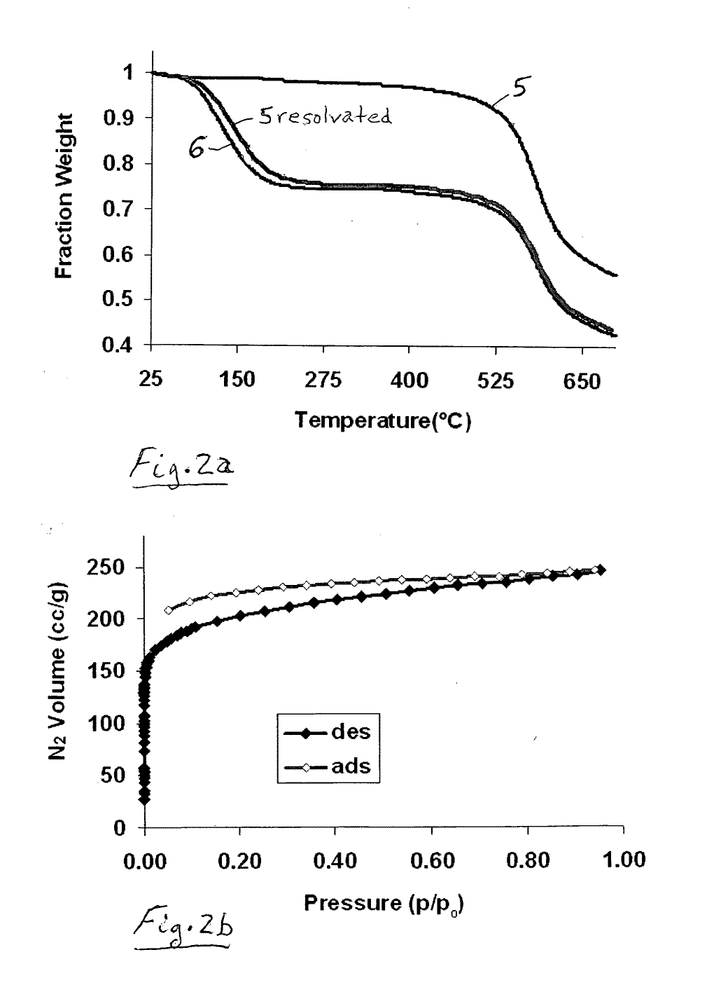

X-ray powder diffraction analysis of the as-synthesized solid polymer 5 revealed no diffraction, implying that 5 is amorphous. SEM images of polymer 5 and 6 (5 heated under a vacuum at 160° C. for 24 h) revealed a series of agglomerates of imperfect, spherically shaped micro- and nanoparticles. The thermal gravimetric analysis (TGA) of 5 and its activated analogue 6, as well as resolvated 6, surprisingly showed stability up to 500° C. (see FIG. 2a). The TGA results imply permanent porosity for polymer 6, because it takes up the same amount of solvent (about 25 wt %) as originally contained in polymer 5. Solid-state ¹³C NMR showed the removal of solvent molecules, as evidenced by the almost complete disappearance of resonances at δ 35 and 30 attributed to DMF. Polyimide connectivity and the presence of derivatives of both building blocks were confirmed by solid-state IR. In polymers 5 and 6, the carbonyl stretch is shifted toward lower energy by approximately 100 cm⁻¹ relative to 4, indicating amide bond formation. N—H stretches are diminished to undetected levels. suggesting essentially complete conversion of starting amine 3. Control experiments with only one of the two reagents present produced no material under the conditions of Scheme 1.

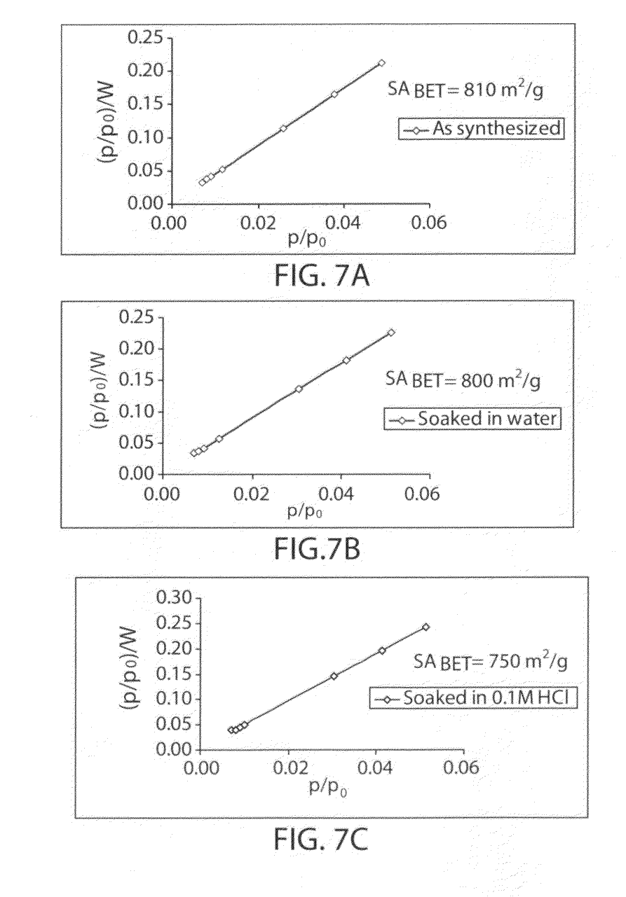

The chemical stability of polymer 5 was evaluated by soaking as-synthesized samples in pure water and in 0.1 M aq. HCl for 24 h. Remarkably, the material fully retained its porosity. The porosity of polymer 6 was quantified via cryogenic adsorption of N₂ (FIG. 2b). The Brunauer-Emmet-Teller (BET) surface area, S.A., was 750 plus or minus 60 m²/g (average of several samples). With CO₂ at 273 K, the measured nonlocal density functional theory (NLDFT) surface area is about 900 m²/g. FIGS. 7a, 7b, and 7c show surface area determination data for polymer 6 in the various conditions indicated.

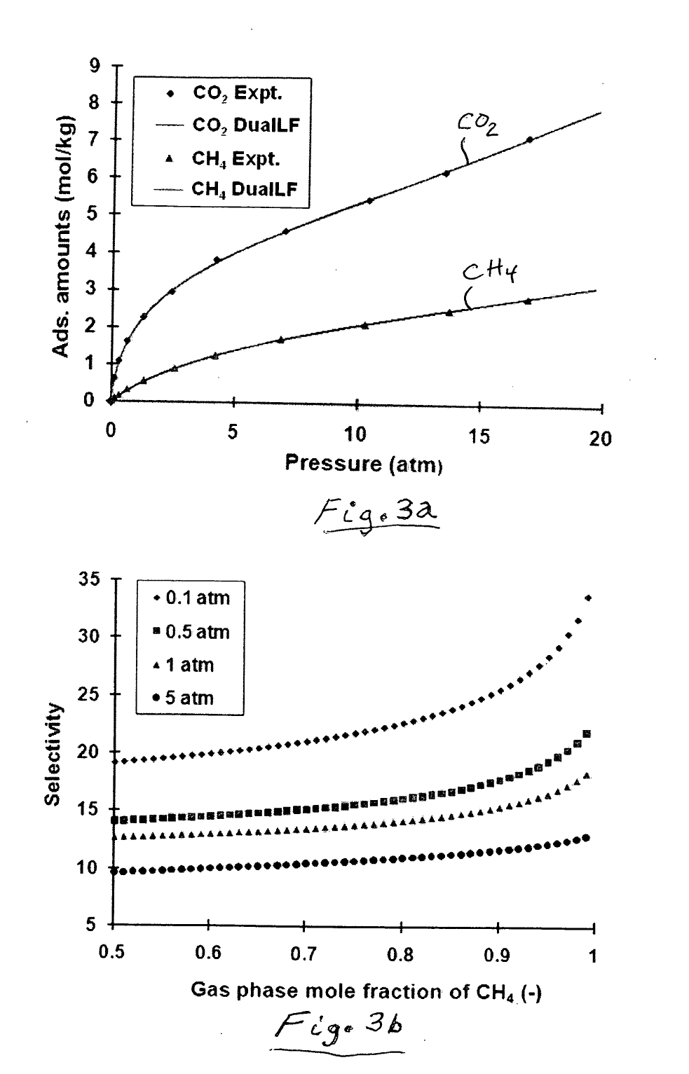

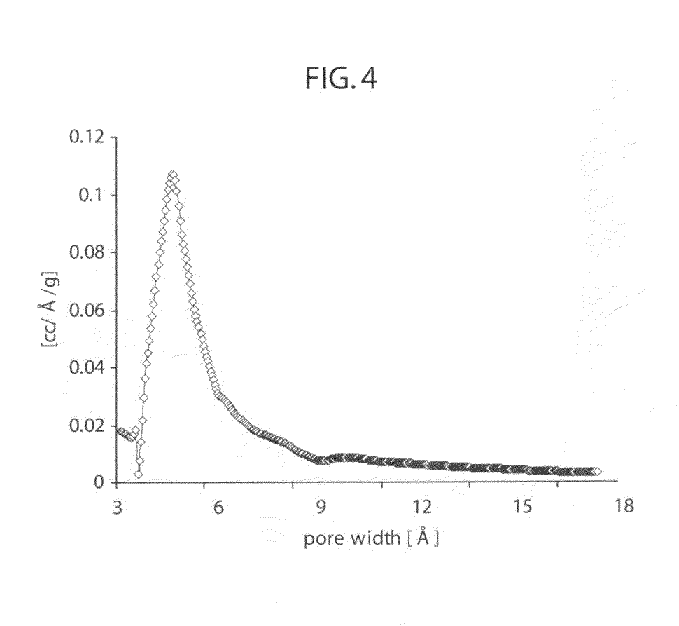

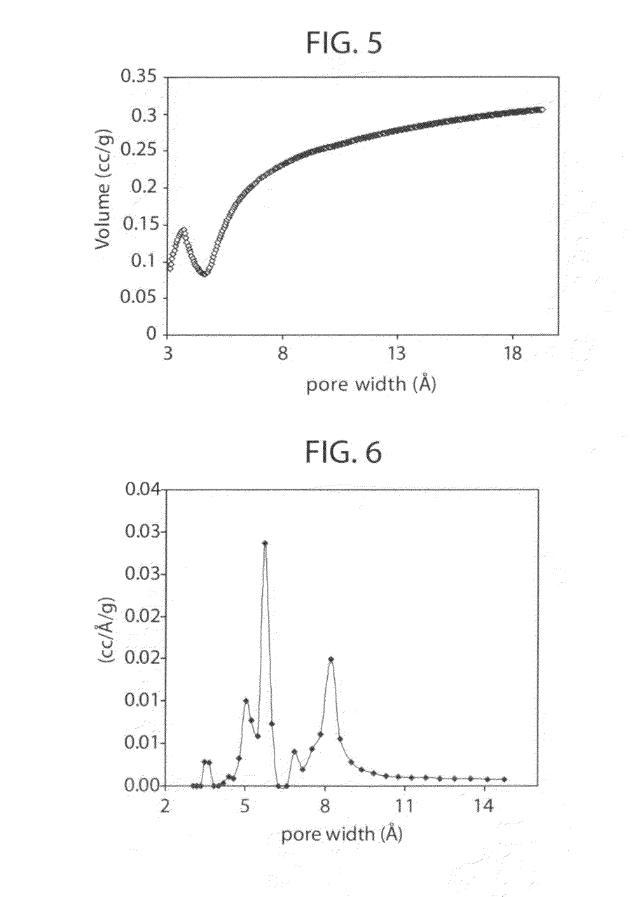

Pore size analysis yielded micro- and ultramicropores of diameter 3.5, 5.2, and 8.2 Angstroms. FIG. 4 shows pore size distribution of polymer 6 obtained by Horvath-Kawazoe (HK) method from the nitrogen isotherm. FIG. 5 shows accumulative pore volume of 6 using the HK method from nitrogen isotherm. FIG. 6 shows pore size distribution of 6 obtained from CO₂ isotherm.

Single-component adsorption isotherms for CO₂ and CH₄ were measured volumetrically for polymer 6 (see FIG. 3a). From the measured pure-component isotherms. the selectivities for CO₂/CH₄ mixtures were calculated using ideal adsorbed solution theory (IAST) (FIG. 3b). Several studies have shown that IAST can be used to effectively predict gas mixture adsorption in zeolites, and MOFs. A dual-site Langmuir-Freundlich model was used to fit the pure isotherms, as shown in FIG. 3a. The fitted isotherm parameters were used to predict the mixture adsorption in 6 by the IAST.

Compound 6 shows increasing CO₂/CH₄ selectivity with decreasing pressure and when the mole fraction of CH₄ (y_{CH4}) approaches unity. In the case of y_{CH4}=0.95, which is a typical feed composition for natural gas purification, the selectivity is in the range of 12-28. Even at y_{CH4}=0.5, high selectivities (9-19) are obtained compared to MOFs (MOF results from GCMC simulations): Cu-BTC (6-10) and MOF-5 (2-3). Experimental and calculated CO₂/CH₄ separations in the most recent study of ZIF materials showed selectivities of 5-10 at 298 K and 800 Torr. Our results are similar to the CO₂/CH₄ selectivities reported for zeolites 13X . However, compared to zeolites, polymer 6 can be regenerated under milder conditions, thus requiring less expenditure of energy. These results indicate that polymer 6 is a promising candidate for the separation and purification of CO₂ from various CO₂/CH₄ mixtures such as natural gas and landfill gas by adsorptive processes.

This Example demonstrates development of a new method for synthesizing new high-area micro- and ultramicroporous organic polymers via amine/anhydride condensation. The first of these new polymer materials simply made from inexpensive precursors, shows outstanding thermal and chemical stability, and exceptional promise for CO₂/CH₄, separation. This amorphous polymer material was shown to be permanently porous and robust, maintaining these properties even when exposed to aqueous and acidic conditions. In addition, polymer 5 exhibited good adsorption selectivity for carbon dioxide over methane.

### Example 2

This example illustrates synthesis of a porous dimide-based organic polymer (POP) post-synthetically reduced with lithium metal to provide a drastic increase in selectivity for carbon dioxide over methane. In the case of polymer 5, this example investigates intercalation of lithium cations between the multiple catenated networks.

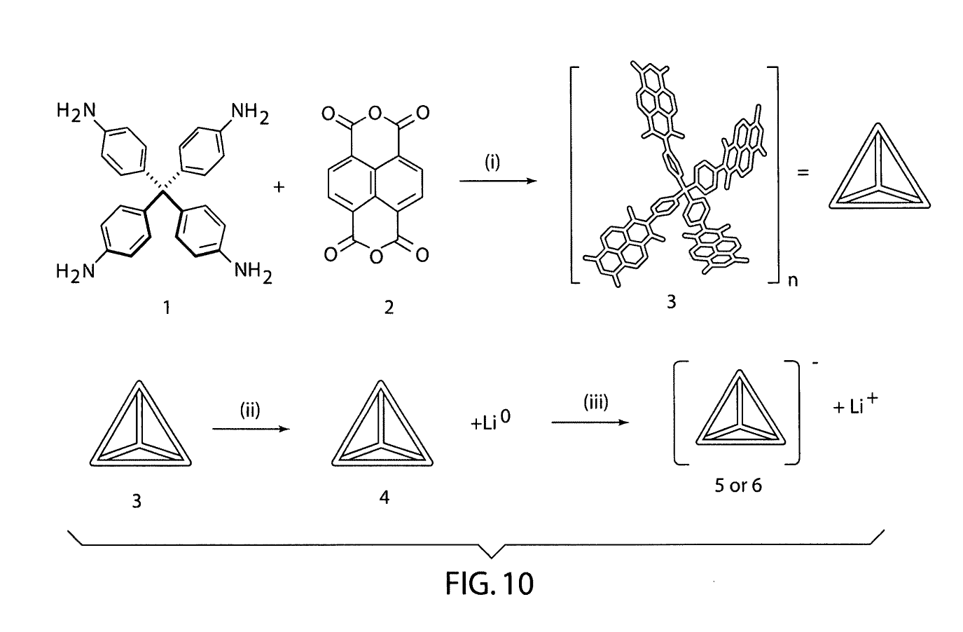

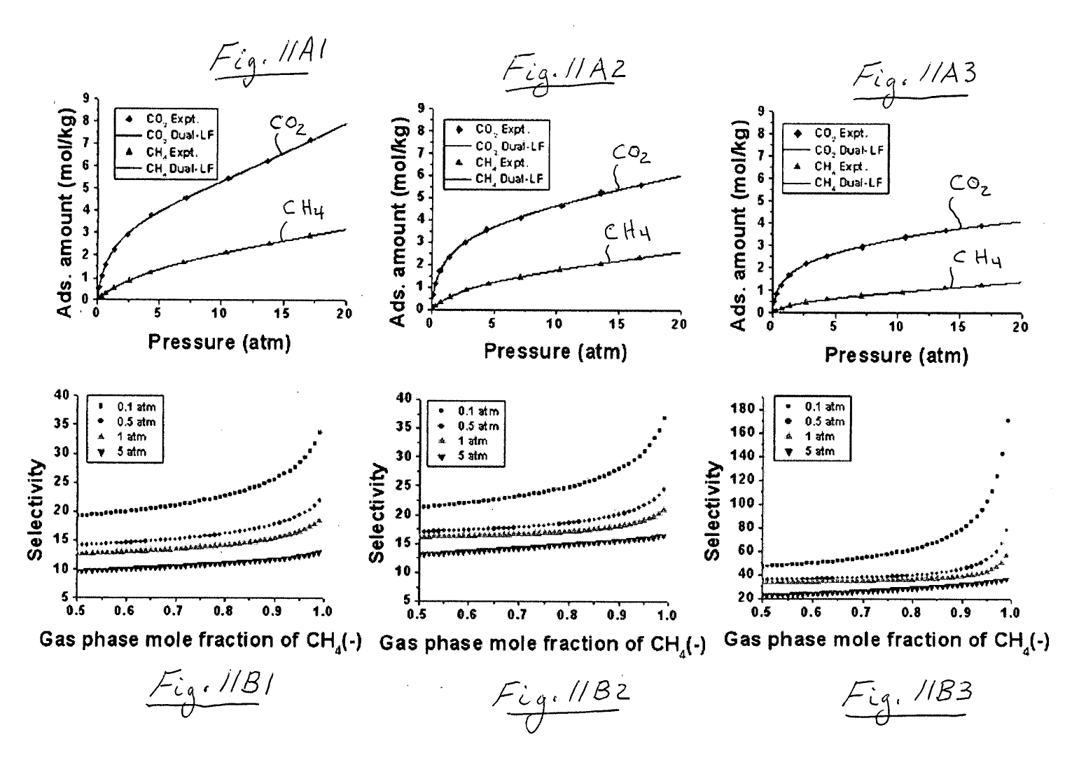

As-synthesized polymer 5 of Example 1 was thermally evacuated under vacuum under Scheme 1′ shown in FIG. 10 wherein polymer 5 of Example 1 is referred to as polymer 3 and is thermally evacuated under vacuum to give polymer 4. In particular, thermal activation of 3 of Scheme 1′ was done under 10⁻⁵ ton dynamic vacuum at 100° C. for 2 hours then 160° C. for 24 hours. The activated sample was then taken into an argon atmosphere glove box. Chemical reduction of polymer 4 (Scheme 1′) was effected by reacting 4 with a solution of lithium metal dissolved in DMF under dry argon gas atmosphere. To make the reductant solution, first a small piece of lithium metal (3.2 mm wire in mineral oil) was cut and rinsed in dry THF to remove mineral oil. Any black oxide was scraped off and a measured amount cut off (1.2 mg for 5; 2.4 mg for 6). The piece of lithium was stirred vigorously for 1 hr in 15 ml dry DMF. To a measured amount of activated 4 (100 mg) the reductant solution was added and allowed to react (10 min for 5 and 15 min for 6). The solution changes from clear to a deep green color and the powder changes from a pale orange color to a dark purple. The powder is filtered on a fine frit and rinsed with 3×5 ml fresh DMF. The reduced samples 5 and 6 are air sensitive and will oxidize if exposed to air. Oxidation is accompanied with a color change back to pale orange. Samples are sealed under argon and again activated under vacuum at 160° C. for 24 hours to remove all DMF before adsorption measurements are taken. Radical formation in the reduced polymer was confirmed with electron paramagnetic resonance (EPR) measurements. Reduced samples of polymer 4 were sealed under inert atmosphere and evacuated under vacuum by heating; care was taken to minimize exposure to oxygen.

Two levels of Li doping were explored, 0.35 and 0.55 lithium atoms per naphthalene diimide linker (5 and 6, respectively in Scheme 1′). Doping levels were controlled by the amount of lithium metal dissolved in DMF as well as the time it was allowed to react. ICP-AES was used to quantify the amount of lithium (see ESI^{t}). Attempts to generate levels higher than 0.55 Li-diimide resulted in loss of material porosity, as evidenced by gas sorption measurements. Thermogravimetric analysis (TGA) of the as-synthesized 3 indicates permanent porosity and shows stability up to 500° C. Porosity of the materials was quantitatively determined by low-pressure adsorption of CO₂. Nitrogen isotherm measurements for 4, 5 and 6 showed no significant uptake of nitrogen for 5 and 6 at 77 K. Surface areas were calculated using non-local density functional theory (NLDFT) methods with CO₂ at 273 K. Overall surface area decreases from 960 m²/g for the as-synthesized material to 750 m²/g and 560 m²/g for 5 and 6, respectively. Partial pore blockage is believed to account for the lower surface areas of the doped materials.

Pure-component isotherms of CO₂ and CH₄ were measured volumetrically on the evacuated samples of 4, 5 and 6 at 298 K, FIGS. 11A1, 11A2, 11A3. Adsorbed CO₂ and CH₄ around 17 bar adhere to the trend of the measured surface areas and decrease with increasing levels of Li-doping, since Li partially reduces the void space within the materials pores. The CO₂/CH₄ selectivities under mixture conditions were predicted from the experimental pure component isotherms using the ideal adsorbed solution theory (IAST). The IAST method is a benchmark tool for determining gas mixture selectivities in zeolites and MOFs. The predicted selectivities at various mixture compositions and pressures are presented in FIGS. 11B1,11B2, 11B3. The selectivity clearly increases with increasing Li-doping. The most striking feature of these figures is the extremely high CO₂/CH₄ selectivity (about 170) of 6 at low pressures.

A typical feed composition for natural gas purification is y_{CH4}=0.95, and a general pressure in the PSA process is around 2 bar (CO₂ partial pressure=0.1 bar). In the CO₂/CH₄ separation from landfill gas, general feed composition and pressure are y_{CH4}=0.5 and 2 bar, respectively (CO₂ partial pressure=1 bar). Extremely high CO₂/CH₄ selectivities are obtained for 5 (17) and 6 (38) in the typical condition of natural gas purification (y_{CH4}=0.95 and 2 bar). Also, 5 and 6 represent very high CO₂/CH₄ selectivities (15 and 30, respectively) in the conditions of landfill gas separation (y_{CH4}=0.5 and 2 bar). These are among the highest selectivities reported for any porous material at similar conditions. Despite the fact that CO₂ uptakes at 298 K and 1 bar (5: 9.1 wt %, 6: 6.6 wt %) are smaller than the values reported for Cu-BTC (17.9 wt %) and zeolite-13X (20.2 wt %), the Li-doped materials (5 and 6) show drastically higher CO₂/CH₄ selectivity than these materials (Cu-BTC: 6 and zeolite-13X: 6) at the condition of landfill gas separation. Additionally, at 298 K and 1 bar CO₂ uptakes are comparable with the value reported for MIL-53 (9.6 wt %) and larger than the values for IRMOF-1 (4.7 wt %), ZIF-100 (4.3 wt %) and MOF-177 (3.5 wt %). These results indicate that 5 and 6 are potential candidates for natural gas purification and landfill gas separation by adsorptive processes.

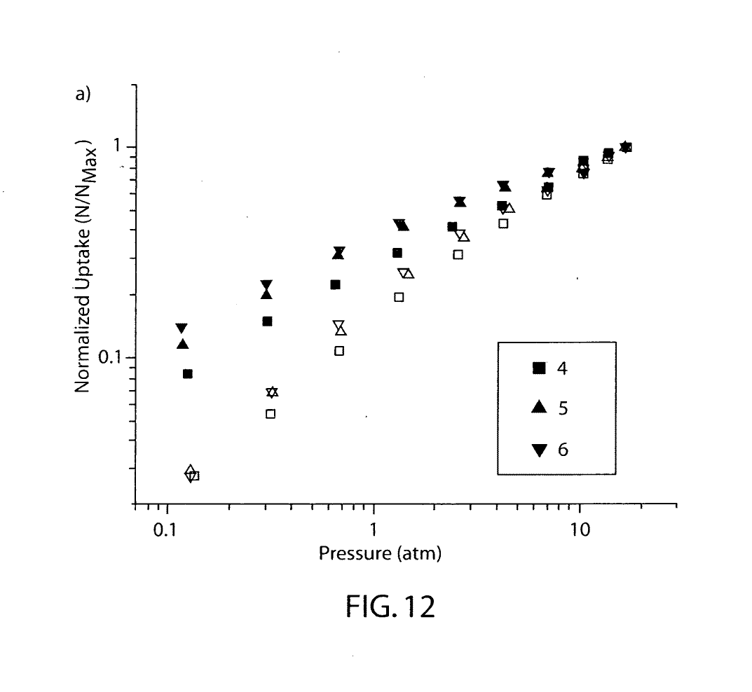

FIG. 12 compares the normalized CO₂ and CH₄ isotherms for 4, 5 and 6 at low pressures. The normalized isotherm was obtained by dividing the adsorbed amount at each pressure (N) by the adsorbed amount at the maximum pressure around 17 bar (Nₘₐₓ). In the case of CO₂ stronger adsorption (as indicated by a higher initial adsorption at low pressure) is observed as the Li-doping amount increases. For CH₄, however, nearly the same relative adsorption is shown independent of the Li-doping amounts. This indicates that Li-doping may induce highly energetic sites within the pores of the material. These could come from chemically reduced ligands or constricted pores. The calculated DFT pore size distributions (CO₂ at 273 K) of 4, 5 and 6 do not suggest any significant change in pore size upon Li-doping. Hence, the strong CO₂ adsorption in 5 and 6 at low pressures likely does not come from the constriction of pores. These energetic sites may also arise from an increased dipole-quadrupolar interaction between CO₂ and the reduced material, but there is little to no effect on the binding of non-polar CH₄. It is evident that the chemically reduced nature of the material leads to the drastic increase in selectivity of polar CO₂ over non-polar CH₄.

This example demonstrates chemical reduction of a permanently porous polymer material with lithium metal. The reduced material retains porosity and demonstrates highly selective adsorption of CO₂ over CH₄. Reduction of similarly structured catenated porous materials with alkali metals could be utilized as a method to increase selective adsorption.

Although the invention has been described above in connection with certain illustrative embodiments, those skilled in the art will appreciate that the invention is not limited to these embodiments and that changes, modifications and the like can be made thereto within the scope of the invention as set forth in the appended claims.

1. A method of separating carbon dioxide from a mixture of carbon dioxide and methane, comprising contacting the mixture and a porous organic polymer material having a three dimensional structure comprising three dimensional building blocks wherein the material selectively adsorbs carbon dioxide.

2. The method of claim 1 wherein the polymer material comprises the building blocks linked by imide linkers.

3. The method of claim 2 wherein the polymer material includes tetrahedral tetra-amino building blocks linked by imide linkers.

4. The method of claim 3 wherein the imide linkers comprise diimide linkers.

5. The method of claim 1 that separates carbon dioxide from natural gas.

6. The method of claim 1 that separates carbon dioxide from landfill gas.

7. The method of claim 1 that uses the pressure swing adsorption process for separation of the mixture.

8. The method of claim 1 including, before the contacting step, chemically reducing the polymer material to increase its selectivity to carbon dioxide.

9. The method of claim 8 wherein the polymer material is reduced using alkali metal.

10. A porous organic polymer that absorbs carbon dioxide comprising tetrahedral building blocks.

11. The polymer of claim 10 wherein the building blocks comprise tetrahedral tetra-amino building blocks.

12. The polymer of claim 10 wherein the tetrahedaral building blocks are linked by imide linkages.

13. The polymer of claim 12 wherein the linkages are diimide linkers.

14. The polymer of claim 12 wherein the diimide linkers comprise napthalene diimide linkers.

15. The polymer of claim 10 wherein the polymer material includes intercalated alkali metal.

16. A porous organic polymer that absorbs carbon dioxide comprising three dimensional building blocks linked by imide linkers.

17. The polymer of claim 16 wherein the building blocks comprise tetrahedral building blocks linked by imide linkages.

18. The polymer of claim 17 wherein the tetrahedral building blocks comprise tetrahedaral tetra-amino building blocks.

19. The polymer of claim 16 wherein the polymer material includes intercalated alkali metal.

20. A porous organic polymer made by condensation of a three dimensional monomer and an imide-linkage forming monomer to form a three dimensional polymer structure that selectively absorbs carbon dioxide.

21. The polymer of claim 20 made by condensation of polyhedral amine-bearing monomer and anhydride-bearing monomer.

22. The polymer of claim 21 wherein the polymer is made by the condensation of a tetrahedral tetra-amino monomer with napthalene dianhydride.

23. The polymer of claim 20 that includes intercalated alkali metal.

24. A method of making a porous organic polymer wherein the polymer is made by condensation of a three dimensional monomer with an imide linkage-forming monomer to form a three dimensional polymer structure that selectively absorbs carbon dioxide.

25. The method of claim 24 wherein the three dimensional monomer is tetrahedral tetra-amine.

26. The method of claim 25 wherein the linkage-forming monomer is napthalene dianhydride.

27. The method of claim 24 further including chemically reducing the polymer to increase is selectively to CO₂.

28. The method of claim 27 wherein the chemically reduction is achieved using alkali metal.
